(19) United States
(12) Patent Application Publication
(10) Pub. No.: US 2016/0237459 A1
(43) Pub. Date: Aug. 18, 2016

(54) Process and Systems for High Solids Fermentation

(71) Applicant: Cellulosic Ethanol Technologies, LLC, Galva, IA (US)

(72) Inventor: Travis BROTHERSON, Holstein, IA (US)

(21) Appl. No.: 15/025,953

(22) PCT Filed: Oct. 15, 2014
(86) PCT No.: PCT/US2014/060701

§ 371 Date: Mar. 30, 2016

Related U.S. Application Data
(60) Provisional application No. 61/891,296, filed on Oct. 15, 2013.

(51) Int. Cl. C12P 7/10; C12P 7/14

(52) U.S. Cl. CPC C12P 7/10; C12P 7/14; C12P 2201/00

(57) ABSTRACT

A process and system for producing ethanol from a biomass feedstock is provided that improves ethanol production by using a biomass feedstock containing high amounts of solids and starch. The process can involve subjecting the biomass feedstock to a primary fermentation to produce a whole stillage and subjecting this whole stillage to a secondary fermentation. The processes and systems described herein can maximize the amount of ethanol produced in ethanol production facilities.

### STATEMENT OF PRIORITY

The present application claims the benefit, under 35 U.S.C. §119(e), of U.S. Provisional Application Ser. No. 61/891,296, filed Oct. 15, 2013, the entire content of which is incorporated by reference herein.

### FIELD OF INVENTION

The present invention relates generally to ethanol production processes and systems. More particularly, the present invention relates generally to fermentation processes utilizing a feedstock containing a high amount of solids.

### BACKGROUND OF THE INVENTION

The production of ethanol for use as a gasoline additive or a straight liquid fuel continues to increase as petroleum costs rise and environmental concerns become more pronounced. Ethanol is generally produced using conventional fermentation processes that convert the starch in plant-based feedstocks into ethanol. However, the yeasts in these conventional fermentation processes are only able to convert limited concentrations of starch in these feedstocks and, therefore, can leave fermentable starch and other valuable sugars in the fermentation byproducts. Consequently, this can result in a reduced yield of ethanol from a bushel of grain and, ultimately, high concentrations of valuable starch leaving the bioprocessing plant in the fermentation byproducts.

Thus, there is a need for a process and system that can maximize the potential of all the starch present in fermentation feedstocks.

### SUMMARY

In one or more embodiments, the present invention concerns a method for producing a biomass-derived product. The method comprises subjecting a whole stillage to fermentation to thereby produce a fermentation product comprising a secondary whole stillage and ethanol, wherein the whole stillage has a starch content of at least 5 weight percent on a dry matter basis.

In one or more embodiments, the present invention concerns a method for producing a biomass-derived product. The method comprising: (a) subjecting a biomass feedstock to a primary fermentation to thereby produce a primary fermentation product comprising a whole stillage and ethanol, wherein the biomass feedstock has a starch content of at least 22 weight percent; and (b) subjecting the whole stillage to a secondary fermentation to thereby produce a secondary fermentation product comprising a secondary whole stillage and ethanol.

In one or more embodiments, the present invention concerns a method for producing a biomass-derived product. The method comprises, consists essentially of, or consists of: (a) subjecting a biomass feedstock to a primary fermentation to thereby produce a primary fermentation product comprising a whole stillage and ethanol, wherein the biomass feedstock has a starch content of at least 20 weight percent and the whole stillage has a starch content of at least 5 weight percent on a dry matter basis; (b) pretreating the whole stillage to thereby produce a pretreated whole stillage; and (c) subjecting the pretreated whole stillage to a secondary fermentation to thereby produce a secondary fermentation product comprising a secondary whole stillage and ethanol.

### BRIEF DESCRIPTION OF THE FIGURES

Embodiments of the present invention are described herein with reference to the following drawing figures, wherein:

FIG. 1 is a flow diagram depicting an exemplary primary fermentation process; and

FIG. 2 is a flow diagram depicting an exemplary secondary fermentation process using the byproducts from the primary fermentation in FIG. 1.

### DETAILED DESCRIPTION

The following detailed description of embodiments of the invention references the accompanying drawings. The embodiments are intended to describe aspects of the invention in sufficient detail to enable those skilled in the art to practice the invention. Other embodiments can be utilized and changes can be made without departing from the scope of the claims. The following detailed description is, therefore, not to be taken in a limiting sense. The scope of the present invention is defined only by the appended claims, along with the full scope of equivalents to which such claims are entitled.

The present invention is generally directed to processes and systems for maximizing ethanol production from a biomass feedstock containing a high amount of solids. More particularly, the present invention is generally directed to processes and systems that involve a primary fermentation step and a secondary fermentation step, which are able to maximize ethanol production from a biomass feedstock containing a high amount of solids. In some embodiments, a high amount of solids for corn can be, for example, about 25% to about 45% or more, and/or any range or value therein (e.g., about 25, 26, 27, 28, 29, 30, 31, 32, 33, 34, 35, 36, 37, 38, 39, 40, 41, 42, 43, 44, 45%).

Additionally, the present invention also provides processes and systems for producing additional ethanol from whole stillage, which can also be processed to form “distiller's grains” or “spent distiller's grains” and other fermentation byproducts. As described below in further detail, due to the use of a biomass feedstock with a high solids content, the processes and systems described herein can allow more fermentable solids to pass through a primary fermentation to a secondary fermentation, which can then be utilized to maximize ethanol production.

In various embodiments described herein, the present invention is directed to a process for producing an alcohol from a biomass feedstock containing a high solids content. As described below in further detail, the process generally comprising: (a) subjecting a biomass feedstock with a high solids content to a primary fermentation to thereby produce a primary fermentation product comprising a whole stillage and at least one alcohol; and (b) subjecting the whole stillage to a secondary fermentation to thereby produce a secondary fermentation product comprising a secondary whole stillage and at least one alcohol.

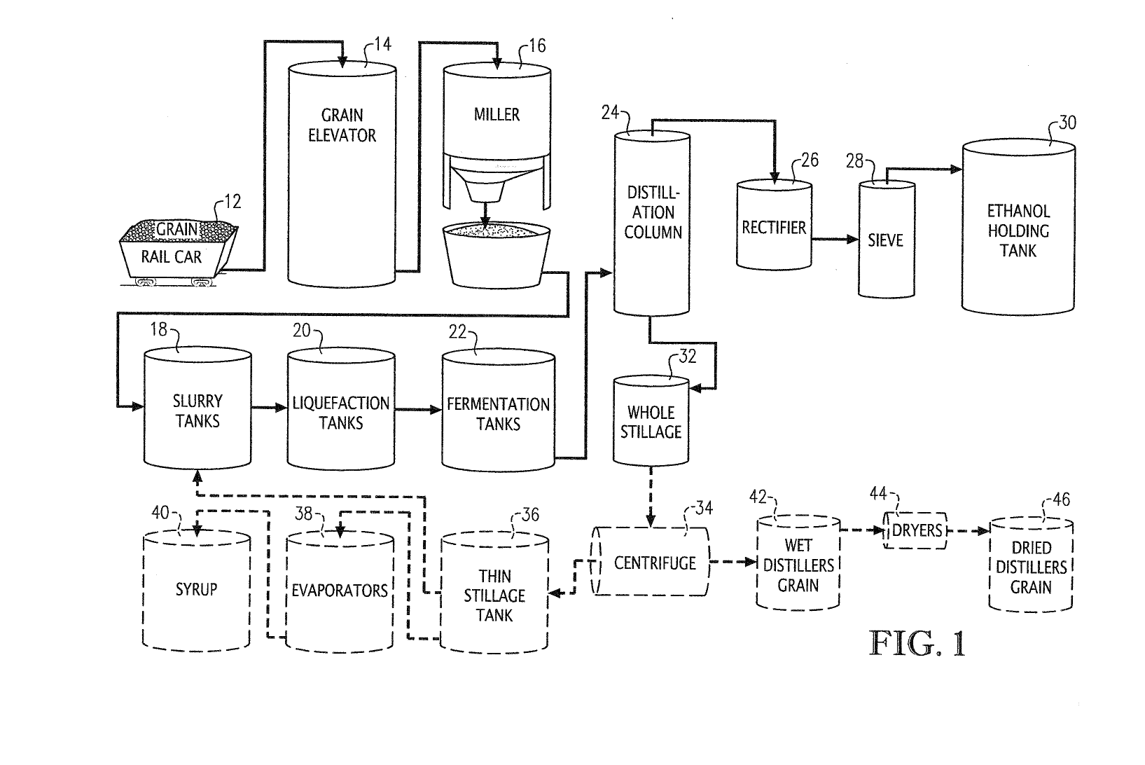

The primary fermentation step is depicted in FIG. 1. However, it should be noted that the primary fermentation process depicted in FIG. 1 can be modified, in whole or part, by other fermentation steps or components without departing from the scope of the present invention. Other fermentation processes are described and illustrated in U.S. Pat. Nos. 6,660,506, 7,527,941, 8,288,138, and 8,409,640 and U.S. Patent Application Publication Nos. 2004/0023349, 2010/0021980, 2012/0045545, 2012/0244591, and 2013/0149764, all of which are incorporated herein by reference in their entireties.

Turning to FIG. 1, a biomass 12 may be delivered to the ethanol production facility by any conventional means known in the art such as, for example, railcars, trucks, or barges. Generally, the biomass feedstock can comprise a grain including, but not limited to, barley, rye, wheat, oats, sorghum, milo, canola, corn, buckwheat, or any combination thereof. As exemplified in FIG. 1, a sufficient supply of the biomass to facilitate the primary fermentation step may be stored in one or more grain elevators 14. In some embodiments, the biomass feedstock can comprise, consist essentially of, consist of at least about 20, 40, or 55 and/or not more than about 90, 75, or 65 weight percent grain. In some embodiments, the grain is ground grain.

Ethanol production can begin by milling or otherwise processing the biomass into a fine powder or flour by a hammer mill or other milling machine 16. The milled biomass can have an average particle size of at least about 100, 500, or 750 μm and/or not more than about 10, 5, or 2 mm, and/or any value or range therein. More particularly, the milled biomass can have an average particle size in the range of about 100 μm to 10 mm, 500 μm to 5 mm, or 750 μm to 2 mm. As used herein, “average particle size” refers to the average width of the milled biomass particles.

In some embodiments, the milled biomass can then be mixed with water in one or more slurry tanks 18 to produce an initial biomass feedstock, which may also be referred to as a “mash.” In some embodiments, the initial biomass feedstock produced in accordance with the aforementioned steps can be a fermentation feedstock that comprises a high amount of solids and starch. For example, in some embodiments, the biomass feedstock can comprise, consist essentially of, or consist of at least about 35, 40, 45, 50, or 60 and/or not more than about 90, 80, 70, or 65 weight percent, and/or any value or range therein, of solids. In further embodiments, the biomass feedstock can comprise, consist essentially of, or consist of in the range of about 35 to 90, 35 to 65, 40 to 80, 45 to 75, 50 to 70, or 60 to 65 weight percent of solids. In particular embodiments, the biomass feedstock can comprise, consist essentially of, or consist of in the range of about 35 to about 65 weight percent of solids, and/or any range or value therein (e.g., about 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65 weight percent).

In some embodiments, the biomass feedstock can comprise, consist essentially of, or consist of at least about 20, 22, 30, 31, 32, 33, 34, 35, 36, 37, 38, 39, 40, 41, 42, 43, 44, or 45 and/or not more than about 95, 90, 80, 75, 70, or 60 weight percent, and/or any value or range therein, of starch. In further embodiments, the biomass feedstock can comprise, consist essentially of, or consist of in the range of about 20 to 95, 22 to 90, 35 to 90, 30 to 80, 35 to 75, 40 to 70, or 45 to 60 weight percent of starch. In some particular embodiments, the biomass feedstock can comprise, consist essentially of, or consist of in the range of about 35 to about 95 weight percent of starch, and/or any range or value therein (e.g., about 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95 weight percent).

It should be noted that all weight percentages described herein are on a dry matter basis of the feedstock unless otherwise noted.

Furthermore, in some embodiments, the biomass feedstock can comprise a significant amount of water from the slurry tanks 18. For example, in some embodiments, the biomass feedstock can comprise, consist essentially of, or consist of at least about 10, 25, 35, 40, or 50 and/or not more than about 90, 85, 75, or 65 weight percent, and/or any value or range therein, of water. In other embodiments, the biomass feedstock can comprise, consist essentially of, or consist of in the range of about 10 to 90, 25 to 85, 35 to 65, 40 to 75, or 50 to 65 weight percent of water. In particular embodiments, the biomass feedstock can comprise, consist essentially of, or consist of in the range of about 35 to about 65 weight percent of water , and/or any range or value therein (e.g., about 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65 weight percent).

In some embodiments, the biomass feedstock for the fermentation can also include recycled components from previous fermentation processes, which can be added to the feedstock in the slurry tanks 18. For example, in some embodiments, the biomass feedstock can comprise a whole stillage and/or thin stillage derived from a previous fermentation process. In further embodiments, the biomass feedstock can comprise, consist essentially of, or consist of at least about 0.5, 1, or 2 and/or not more than about 20, 10, or 5 weight percent, and/or any value or range therein, of the solids from a thin stillage recycled from a previous fermentation process. In still further embodiments, the biomass feedstock can comprise, consist essentially of, or consist of in the range of about 0.5 to 20, 1 to 10, or 2 to 5 weight percent of the solids from a thin stillage derived from a previous fermentation process. In some particular embodiments, the biomass feedstock can comprise, consist essentially of, or consist of in the range of about 0.5 to about 20 weight percent of the solids from a thin stillage recycled from a previous fermentation process, and/or any range or value therein (e.g., about 0.5, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20 weight percent).

Furthermore, in some embodiments, at least 5, 20, or 40 and/or not more than about 95, 80, or 60 percent of the water in the biomass feedstock can be derived from the thin stillage. In some embodiments, the solids in the thin stillage can make up at least about 0.5, 1, or 2 and/or not more than about 20, 10, or 5 weight percent of the biomass feedstock.

As exemplified in FIG. 1, the initial biomass feedstock can then be mixed with enzymes in a liquefaction tank 20 and held in this tank for a sufficient amount of time to enable the enzymes to begin hydrolyzing the starch in the feedstock into fermentable sugars. In some embodiments, the amount of enzyme activity in this step, especially if gluco-amylase is utilized, may be maintained at lower levels in order to leave more long chain sugars in the biomass feedstock. In some embodiments, an enzyme useful with this invention can include but is not limited to a protease, alpha-amylase, gluco-amylase, xylanase, cellobiohydrolase, beta-glucosidase cellulase, amylase, hemicellulase, or any combination thereof In some embodiments, the enzymes may be added at a concentration in the range of about 0.001 to 0.5, 0.005 to 0.3, or 0.01 to 0.2 weight percent based on the dry matter of the solids. The temperatures and conditions for this treatment can vary depending on the type of enzymes used as known in the art. During this treatment, in some embodiments, at least 10, 20, or 30 and/or not more than 90, 70, or 60 percent, and/or any value or range therein, of the starch present in the biomass feedstock can be hydrolyzed into long chain sugars. In further embodiments, this treatment can hydrolyze in the range of 10 to 90, 20 to 70, or 30 to 60 percent of the starch into long chain sugars. In particular embodiments, at least about 30 to about 90 percent, and/or any value or range therein, of the starch present in the biomass feedstock can be hydrolyzed into long chain sugars , and/or any range or value therein (e.g., about 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65 66, 67, 68, 69, 70, 71, 72, 73, 74, 75, 76, 77, 78, 79, 80, 81, 82, 83, 84, 85, 86, 87, 88, 89, 90 weight percent).

Turning again to exemplary FIG. 1, the treated biomass feedstock can be introduced into one or more fermentation tanks 22 where one or more yeast types can be added to facilitate fermentation. In some embodiments, the added yeast can comprise Saccharomyces cerevisiae. In some embodiments, this fermentation step can produce a primary fermentation product that can comprise alcohols and various other solid and liquid byproducts. The primary fermentation product may also be commonly referred to as “beer” by those skilled in the art. In some embodiments, the primary fermentation step described herein can convert at least about 50, 75, 85, or 95 percent, and/or any value or range therein, of the starch originally found in the biomass into the primary fermentation product.

In further embodiments, the primary fermentation can occur over a time period in the range of 12 to 150, 24 to 130, or 36 to 110 hours, and/or any value or range therein. In some embodiments, depending on the type of yeasts used, the primary fermentation can generally occur at a temperature in the range of 50 to 140, 70 to 120, or 80 to 97° F., and/or any value or range therein. In further embodiments, the primary fermentation can occur at a pH in the range of about 3 to 8, 3.5 to 6, or 4 to 5, and/or any value or range therein.

In order to compensate for the possible high viscosity of the feedstock due to its high solids content, in some embodiments, the amount of alpha-amylase enzymes that can be added to the feedstock before fermentation during the liquefaction step and/or during fermentation itself can be greater than the amount that is typically used in the art. Consequently, these enzymes can break down some of the starch in the feedstock, thereby reducing the viscosity of the biomass feedstock. Thus, the feedstock can be easier to move throughout the system depicted, for example, in FIG. 1. In representative embodiments, the alpha-amylase can be derived solely from the grain used as the biomass feedstock, which has been genetically modified to express higher quantities of this enzyme. In such embodiments, additional alpha-amylase can be added or withheld. In some embodiments, where alpha-amylase is added, it may be added at a concentration in the range of about 0.001 to 0.5, 0.005 to 0.3, or 0.01 to 0.2, and/or any value or range therein, weight percent based on the dry matter of the solids.

As noted above, the primary fermentation product can comprise multiple types of alcohols and other various solid and liquid byproducts. However, ethanol is usually the major component and the most important commercial product produced during the primary fermentation process. In some embodiments, the primary fermentation product can comprise, consist essentially of, or consist of at least about 7, 10, 13, or 15 and/or not more than about 40, 35, 30, or 25 weight percent, and/or any value or range therein, of ethanol. In further embodiments, the primary fermentation product can comprise, consist essentially of, or consist of in the range of about 7 to 40, 10 to 35, 13 to 30, 7 to 25, 15 to 25 weight percent of ethanol. In particular embodiments, primary fermentation product can comprise, consist essentially of, or consist of at least about 7 to about 25 weight percent, and/or any value or range therein, of ethanol (e.g., about 7, 8, 9, 10, 11, 12, 13, 14, 15, 16, 17, 18, 19, 20, 21, 22, 23, 24, 25 weight percent). In still further embodiments, the primary fermentation can produce at least about 1.3, 2.1, 2.25, 2.4, or 2.65 and/or not more than about 3.8, 3.5, 3.3, 3.1, or 2.9 gallons, and/or any value or range therein, of ethanol per bushel of grain. In some embodiments, the primary fermentation can produce in the range of about 1.3 to 3.8, 2.1 to 3.5, 2.25 to 3.3, 2.4 to 3.1, 2.65 to 2.9 gallons of ethanol per bushel of grain. In particular embodiments, the primary fermentation can produce at least about 1.3 to about 2.9 gallons, and/or any value or range therein, of ethanol per bushel of grain (e.g., about 1.3, 1.4, 1.5, 1.6, 1.7, 1.8, 1.9, 2, 2.1, 2.2, 2.3, 2.4, 2.5 2.6, 2.7, 2.8, 2.9 gallons).

In some embodiments, other byproducts in the primary fermentation product can include, for example, glycerol, acetic acid, lactic acid, and carbon dioxide. In some embodiments, the primary fermentation product can comprise, consist essentially of, or consist of at least about 0.1, 0.5, or 1 and/or not more than about 5, 3, or 2 weight percent, and/or any value or range therein, of glycerol. In further embodiments, the primary fermentation product can comprise, consist essentially of, or consist of in the range of about 0.1 to 5, 0.5 to 3, or 1 to 2 weight percent of glycerol. In particular embodiments, the primary fermentation product can comprise, consist essentially of, or consist of at least about 0.5 to about 3 weight percent, and/or any value or range therein, of glycerol (e.g., about 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5 2.6, 2.7, 2.8, 2.9, 3 weight percent). In some embodiments, the primary fermentation product can comprise, consist essentially of, or consist of at least about 0.001, 0.005, or 0.01 and/or not more than about 0.5, 0.3, or 0.2 weight percent, and/or any value or range therein, of acetic acid. In further embodiments, the primary fermentation product can comprise, consist essentially of, or consist of in the range of about 0.001 to 0.5, 0.005 to 0.3, or 0.01 to 0.2 weight percent of acetic acid. In particular embodiments, the primary fermentation product can comprise, consist essentially of, or consist of at least about 0.001 to about 0.5 weight percent, and/or any value or range therein, of acetic acid (e.g., about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5 weight percent). In some embodiments, the primary fermentation product can comprise, consist essentially of, or consist of at least about 0.001, 0.005, or 0.01 and/or not more than about 2, 1.5, or 1 weight percent, and/or any value or range therein, of lactic acid. In further embodiments, the primary fermentation product can comprise, consist essentially of, or consist of in the range of about 0.001 to 2, 0.005 to 1.5, or 0.01 to 1 weight percent of lactic acid. In particular embodiments, the primary fermentation product can comprise, consist essentially of, or consist of at least about 0.005 to about 2 weight percent, and/or any value or range therein, of lactic acid (e.g., about 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2 weight percent). It should be noted that the above weight percentages are based on the total weight of the fermentation product unless otherwise noted.

As exemplified in FIG. 1, the primary fermentation product can be transferred to one or more distillation columns 24, which are also known in the art as “beer strippers,” in order to separate the alcohols, especially ethanol, from the solids and other liquids. In some embodiments, the alcohol can exit the top of these columns 24 and can be transferred to one or more rectifiers 26 to further remove moisture from the alcohol. In further embodiments, the alcohol may also be passed to one or more molecular sieves 28 in order to remove even more moisture. In still further embodiments, the final alcohol can then be transferred to one or more ethanol holding tanks 30 where it may be denatured before use as a fuel or fuel additive.

In some embodiments, the distillation of the primary fermentation product can be influenced by the amount of short chain sugars present in the product. In particular, distillation can be negatively impacted if there are too many short chain sugars present in the primary fermentation product. Thus, in some embodiments, the whole stillage of the primary fermentation product (e.g., primary whole stillage) can comprise, consist essentially of, or consist of less than about 10, 5, 3, or 1 weight percent of sugars, and/or any range or value therein, having a degree of polymerization of not more than about 20, 15, 12, 10, or 4, and/or any range or value therein.

The liquid and solid mixture that remains in distillation columns 24 after the alcohol has been removed is commonly referred to as “whole stillage” or simply “stillage.” The mixture can also be commonly referred to as “distiller's grains” or “spent distiller's grains.” In some embodiments, the whole stillage generally can settle to the bottom of the distillation columns 24 and can then be transferred to one or more whole stillage holding tanks 32.

In some embodiments, the primary whole stillage can comprise, consist essentially of, or consist of at least about 10, 12, 20, or 25 and/or not more than about 60, 55, 50, or 45 weight percent, and/or any range or value therein, of solids. In other embodiments, the whole stillage can comprise, consist essentially of, or consist of in the range of about 10 to 60, 10 to 65, 12 to 55, 20 to 50, or 25 to 45 weight percent solids. In particular embodiments, the whole stillage can comprise, consist essentially of or consist of at least about 10 to about 45 weight percent, and/or any range or value therein, of solids (e.g., about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65 weight percent). In further embodiments, the whole stillage can comprise, consist essentially of, or consist of at least about 5, 15, 25, or 40 and/or not more than about 90, 70, 60, or 50 weight percent, and/or any range or value therein, of water. In still further embodiments, the whole stillage can comprise, consist essentially of, or consist of in the range of about 5 to 90, 15 to 70, 25 to 60, 45 to 90, or 40 to 50 weight percent of water. In particular embodiments, the whole stillage can comprise, consist essentially of, or consist of about 45 to about 90 weight percent, and/or any range or value therein, of water (e.g., about 45, 46, 47, 48, 49, 50, 51, 52, 53, 54, 55, 56, 57, 58, 59, 60, 61, 62, 63, 64, 65 66, 67, 68, 69, 70, 71, 72, 73, 74, 75, 76, 77, 78, 79, 80, 81, 82, 83, 84, 85, 86, 87, 88, 89, 90 weight percent).

A primary whole stillage produced by the primary fermentation step can have a number of uses. For example, in some embodiments, a whole stillage may be optionally passed through one or more centrifuges 34, which can separate it into a stream of thin stillage and a stream of wet distiller's grain.

In some embodiments, thin stillage can be mostly liquid but may also contain a small amount of solid materials. In some embodiments, thin stillage may be held in one or more tanks 36 and can be returned to the slurry tanks 18 or some other part of the fermentation process that requires water. In further embodiments, the thin stillage can comprise, consist essentially of, or consist of at least about 50, 75, or 85 and/or not more than about 99, 95, or 90 weight percent, and/or any range or value therein, of water. In some embodiments, the thin stillage can comprise, consist essentially of, or consist of in the range of about 50 to 99, 75 to 95, 75 to 99, or 85 to 90 weight percent of water. In particular embodiments, the primary whole stillage can comprise, consist essentially of, or consist of about 75 to about 99 weight percent, and/or any range or value therein, of water (e.g., about 75, 76, 77, 78, 79, 80, 81, 82, 83, 84, 85, 86, 87, 88, 89, 90, 91, 92, 93, 94, 95, 96, 97, 98, 99 weight percent). In further embodiments, the thin stillage can comprise, consist essentially of, or consist of at least about 1, 3, or 5 and/or not more than about 20, 15, or 10 weight percent, and/or any range or value therein, of solids. In still further embodiments, the thin stillage can comprise, consist essentially of, or consist of in the range of about 1 to 20, 3 to 15, 3 to 20, or 5 to 10 weight percent of solids. In particular embodiments, the primary whole stillage can comprise, consist essentially of, or consist of about 3 to about 20 weight percent, and/or any range or value therein, of solids (e.g., about 3, 4, 5, 6, 7, 8, 9, 10, 11, 12, 13, 14, 15, 16, 17, 18, 19, 20 weight percent).

In some embodiments, some or all of the thin stillage may also be transferred to one or more evaporators 38 to produce an evaporated thin stillage, which is commonly referred to as “syrup.” In further embodiments, the syrup may be held in one or more tanks 40 and be used as an animal feed additive.

In some embodiments, the wet distiller's grain, which can also be referred to as “wetcake,” may be held in storage facilities 42 and sold as a livestock feed. In further embodiments, some of the wet distiller's grain may also be transferred to one or more dryers 44 to remove liquid therefrom to produce dried distiller's grain, which may also be stored in one or more tanks 46 and used as livestock feed. In additional embodiments, some of the syrup can be dried with the wet distiller's grains to produce dried distillers grains with solubles (“DDGS”).

In some embodiments, unlike conventional fermentation processes, the processes and systems described herein do not discard the whole stillage, but can use this byproduct to produce additional ethanol. In some embodiments, the whole stillage and/or wet distiller's grain can be subjected to a secondary fermentation step in order to maximize ethanol production. Thus, during a second fermentation, additional ethanol can be produced from any residual starch leftover in the whole stillage and/or the fiber portions in the whole stillage. One advantage of employing the secondary fermentation described herein is that it can be utilized to maximize ethanol production from the byproducts derived from the primary fermentation step rather than just using the byproducts as animal feed.

Since the initial biomass feedstock can contain high amounts of solids and starch as noted above, in some embodiments, the whole stillage (e.g., primary whole stillage) used in the secondary fermentation step described herein can contain more residual starch compared to conventional whole stillages. For example, in some embodiments, the whole stillage can comprise, consist essentially of, or consist of at least about 5, 10, 15, or 20 and/or not more than about 80, 60, 50, or 40 weight percent, and/or any range or value therein, of starch on a dry matter basis. Thus, in some embodiments, the whole stillage can comprise, consist essentially of, or consist of in the range of about 5 to 80, 5 to 60, 10 to 60, 15 to 50, or 20 to 40 weight percent of starch on a dry matter basis. In particular embodiments, the whole stillage can comprise, consist essentially of, or consist of at least about 5 to about 60 weight percent, and/or any range or value therein, of solids (e.g., about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60 weight percent).

Furthermore, although a primary fermentation step can convert a significant portion of the starch in the biomass feedstock, in some embodiments, a good portion of the starch can remain in the whole stillage. For example, in some embodiments, the biomass feedstock used in the primary fermentation and the produced primary whole stillage can comprise, consist essentially of, or consist of a starch ratio of at least about 2:1, 3:1, 4:1, or 5:1 and/or not more than about 50:1, 25:1, 20:1, or 10:1, and/or any range or value therein. In further embodiments, the biomass feedstock used in the primary fermentation and the produced whole stillage can comprise, consist essentially of, or consist of a starch ratio in the range of 2:1 to 50:1, 3:1 to 25:1, 4:1 to 20:1, or 5:1 to 10:1. In particular embodiments, the biomass feedstock used in the primary fermentation and the produced whole stillage can comprise, consist essentially of, or consist of a starch ratio in the range of 2:1 to 50:1, and/or any range or value therein (e.g., a starch ratio of about 2:1, 3:1, 4:1, 5:1, 6:1, 7:1, 8:1, 10:1, 11:1, 12.1, 13:1, 14:1, 15:1, 16:1, 17:1, 18:1, 19:1, 20:1, 21:1, 22:1, 23:1, 24:1, 25:1, 26:1, 27:1, 28:1, 29:1, 30:1, 31:1, 32:1, 33:1, 34:1, 35:1, 36:1, 37:1, 38:1, 39:1, 40:1, 41:1, 42:1, 43:1, 44:1, 45:1, 46:1, 47:1, 48:1, 49:1, 50:1). Due to the larger amount of residual starch found in the whole stillage described herein, more ethanol can be produced in the secondary fermentation.

Moreover, in some embodiments, the secondary fermentation step can be used to convert the various fiber components of the whole stillage into additional ethanol. Since whole stillage is generally the byproduct of the fermentation of corn or other cereal grain, it can contain a sizable fraction of fiber. All fiber is made up of hemicellulose, cellulose, and lignin. Cellulose is comprised of glucose molecules, the same as in starch, but the linkages in cellulose make it more difficult to break down into individual glucose molecules than in starch. Hemicellulose contains a mixture of sugars and is generally easier to breakdown than cellulose. Lignin and/or pectin functions as a binder and cannot generally be broken down into fermentable sugars. Thus, in some embodiments, the processes of the present invention can also include steps for converting both the hemicellulose and cellulose portions of the whole stillage into sugars that may be fermented into ethanol.

In some embodiments, prior to the secondary fermentation, the primary whole stillage can be subjected to (1) prolonged soaking in the liquefaction tanks, (2) heating in the distillation columns, and/or (3) chemical reactions from the various chemical additives added during the primary fermentation. These steps can help facilitate the breakdown of the fibers in the primary whole stillage and make them easier to convert into ethanol and other useful byproducts during the secondary fermentation step.

In some embodiments, the primary whole stillage can comprise, consist essentially of, or consist of at least about 5, 8, 10, or 12 and/or not more than about 30, 25, 20, or 17 weight percent, and/and/or any range or value therein, of cellulose on a dry matter basis. In some embodiments, the primary whole stillage can comprise, consist essentially of, or consist of at least about 5, 8, 10, or 12 and/or not more than about 30, 25, 20, or 17 weight percent, and/or any range or value therein, of cellulose on a dry matter basis. In other embodiments, the primary whole stillage can comprise, consist essentially of, or consist of in the range of about 5 to about 30 weight percent, and/or any range or value therein, of cellulose on a dry matter basis (e.g., about 5, 6, 7, 8, 9, 10, 11, 12, 13, 14, 15, 16, 17, 18, 19, 20, 21, 22, 23, 24, 25, 26, 27, 28, 29, 30 weight percent). In particular embodiments, the cellulose content of the whole stillage can be calculated by subtracting the acid detergent fibers content from the neutral detergent fibers content. Furthermore, in some embodiments, the primary whole stillage can comprise, consist essentially of, or consist of at least about 5, 8, 10, or 12 and/or not more than about 30, 25, 20, or 17 weight percent, and/or any range or value therein, of hemicellulose on a dry matter basis. In further embodiments, the primary whole stillage can comprise, consist essentially of or consist of in the range of about 5 to 30, 8 to 25, 10 to 20, or 12 to 17 weight percent of hemicellulose on a dry matter basis. In other embodiments, the primary whole stillage can comprise, consist essentially of, or consist of in the range of about 5 to about 30 weight percent, and/or any range or value therein, of hemicellulose on a dry matter basis (e.g., about 5, 6, 7, 8, 9, 10, 11, 12, 13, 14, 15, 16, 17, 18, 19, 20, 21, 22, 23, 24, 25, 26, 27, 28, 29, 30 weight percent).

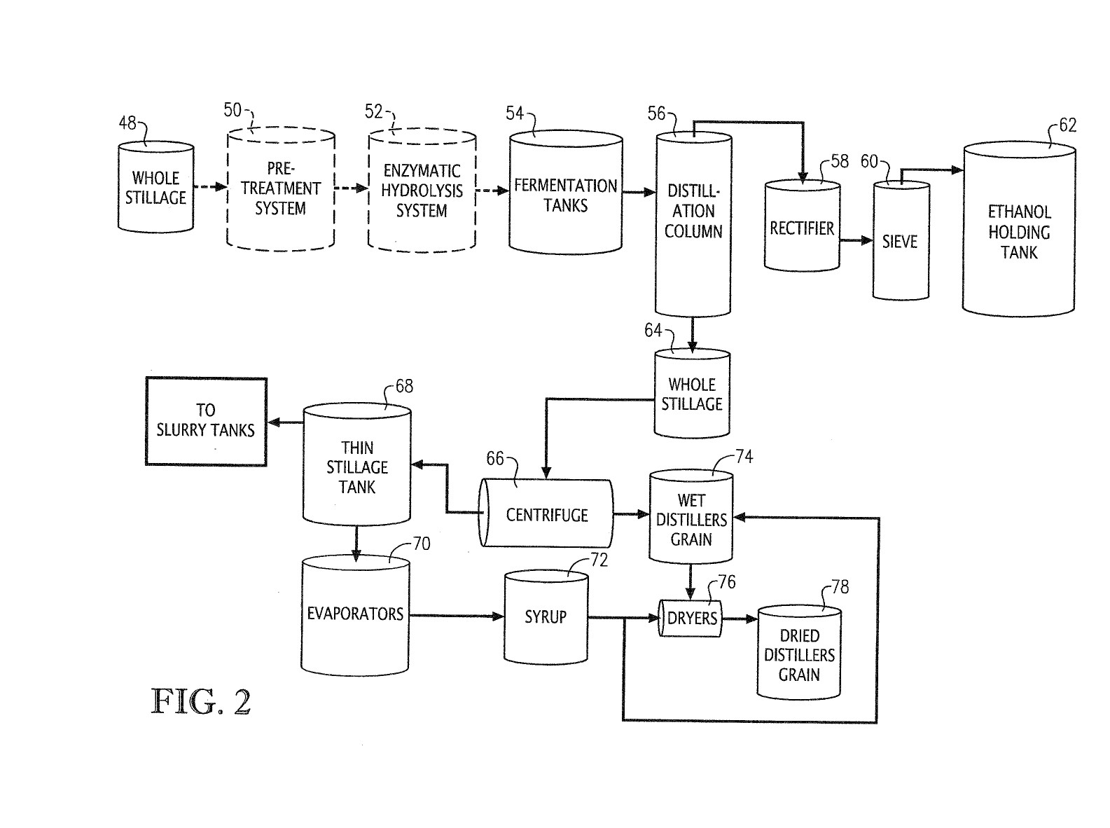

The secondary fermentation process is exemplified in FIG. 2. It should be noted that the secondary fermentation process depicted in FIG. 2 can be modified, in whole or part, by other fermentation steps or components without departing from the scope of the present invention.

In some embodiments, as shown in FIG. 2, the secondary fermentation process can utilize the primary whole stillage 48 produced in the primary fermentation as its primary feedstock and further convert the whole stillage into ethanol and other useful byproducts. In further embodiments, the secondary fermentation process can also utilize the wet distiller's grain produced in the primary fermentation.

Prior to fermentation, the whole stillage can optionally be subjected to one or more pretreatments in a pretreatment system 50. The pretreatments can include, but are not limited to, steam explosion, acid hydrolysis, alkaline treatment, torrefaction, drying, grinding, soaking, or combinations thereof. The grinding can include, but is not limited to, wet milling or dry milling. These pretreatments can be utilized to break down some of the starch, cellulose and/or hemicellulose within the whole stillage into fermentable sugars. In some embodiments, pretreating the cellulose and hemicellulose portions of the whole stillage can make these components more susceptible to degradation into fermentable sugars, especially after these components have been allowed to soak in the primary fermentation and been subjected to distillation. Thus, in some embodiments, this can allow for a greater yield of sugars from the fiber components.

In one or more embodiments, the pretreatment can comprise heating the whole stillage and subjecting it to high pressures. The optimum temperatures of this pretreatment can depend on a variety of factors including, for example, upstream treatments and retention times, downstream retention times, the whole stillage's pH value, and the enzyme treatments described herein. In some embodiments, the heating can be performed in a hydro-heater, wherein high pressure steam can be injected into the whole stillage and thereby increasing its temperature to a range of about 215° F. to 260° F., with higher temperatures generally being preferable. In other embodiments, higher temperatures up to about 300° F. may be even more beneficial. In some embodiments, the whole stillage can be held at these elevated temperatures (e.g., about 215° F. to about 300° F.) and pressures (e.g., pressures above boiling point, e.g., about 30 psi) for at least about 5 seconds and generally not more than about 20 minutes, and/or any range or value therein. Heating by steam injection can be beneficial because it results in cavitation of the whole stillage, which further disrupts the structure of the fiber within the whole stillage thereby aiding the subsequent processing of the whole stillage. In further embodiments, additional steam injection steps can be added to further break down the fiber. The number of steam injection steps is a trade-off between energy use and yield and product quality.

In some embodiments, following the above heat treatment in, for example, a steam injection unit, the whole stillage can be subjected to a steam explosion that involves rapidly dropping the pressure to thereby cause the whole stillage to boil and flash off steam. This rapid boiling can cause further rupturing of the fiber structures within the whole stillage, thereby further exposing the cellulose and hemicellulose within the fibers.

In some embodiments, the pretreatment can comprise adding an acid to the whole stillage to decrease its pH level; heating and pressurizing the whole stillage; holding the whole stillage under pressure and heat; removing pressure from the whole stillage to cause flashing; and cooling the whole stillage before the enzymes are added.

Additional pretreatment processes are further described in U.S. Patent Application Publication Nos. 2012/0045545, 2013/0149763, and 2013/0149750, the disclosures of which are incorporated herein by reference in their entireties.

The pretreatments described herein can be used to break down at least a portion of the starch, cellulose, and/or hemicellulose in the whole stillage into fermentable sugars. In some embodiments where the whole stillage has been subjected to a pretreatment, the pretreated whole stillage can comprise, consist essentially of, or consist of at least about 1, 3, 5, or 10 and/or not more than about 50, 40, 30, or 20 weight percent, and/or any range or value therein, of starch on a dry matter basis. In some embodiments, the pretreated whole stillage can comprise, consist essentially of, or consist of in the range of about 1 to 50, 3 to 40, 5 to 30, or 10 to 20 weight percent, and/or any range or value therein, of starch on a dry matter basis. In particular embodiments, where the whole stillage has been subjected to a pretreatment, the pretreated whole stillage can comprise, consist essentially of, or consist of at least about 1 to about 50 weight percent, and/or any range or value therein, of starch on a dry matter basis (e.g., about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 weight percent). In further embodiments, the pretreated whole stillage can comprise, consist essentially of, or consist of at least about 2, 5, 7, or 10 and/or not more than about 25, 20, 18, or 15 weight percent, and/or any range or value therein, of cellulose on a dry matter basis. In still further embodiments, the pretreated whole stillage can comprise, consist essentially of, or consist of in the range of 2 to 25, 5 to 20, 7 to 18, or 10 to 15 weight percent of cellulose on a dry matter basis. In particular embodiments, the pretreated whole stillage can comprise, consist essentially of, or consist of at least about 2 to about 30 weight percent, and/or any range or value therein, of cellulose on a dry matter basis (e.g., about 2, 3, 4, 5, 6, 7, 8, 9, 10, 11, 12, 13, 14, 15, 16, 17, 18, 19, 20, 21, 22, 23, 24, 25, 26, 27, 28, 29, 30 weight percent). In some embodiments, the pretreated whole stillage can comprise, consist essentially of, or consist of at least about 2, 5, 7, or 10 and/or not more than about 25, 20, 18, or 15 weight percent, and/or any range or value therein, of hemicellulose on a dry matter basis. In further embodiments, the pretreated whole stillage can comprise, consist essentially of, or consist of in the range of 2 to 25, 5 to 20, 7 to 18, or 10 to 15 weight percent of hemicellulose on a dry matter basis. In particular embodiments, the pretreated whole stillage can comprise, consist essentially of, or consist of in the range of about 2 to about 25 weight percent, and/or any range or value therein, of hemicellulose on a dry matter basis (e.g., 2, 3, 4, 5, 6, 7, 8, 9, 10, 11, 12, 13, 14, 15, 16, 17, 18, 19, 20, 21, 22, 23, 24, 25 weight percent).

As noted above, the pretreatments can be utilized to breakdown and weaken some of the solids within the whole stillage in order derive fermentable sugars therefrom. Thus, in some embodiments, the pretreated whole stillage can comprise, consist essentially of, or consist of at least about 5, 7, 10, or 15 and/or not more than about 50, 40, 25, or 20 weight percent, and/or any range or value therein, of solids. In some embodiments, the pretreated whole stillage can comprise, consist essentially of, or consist of in the range of about 5 to 50, 10 to 40, 7 to 40, 10 to 25, or 15 to 20 weight percent of solids. In particular embodiments, the pretreated whole stillage can comprise, consist essentially of, or consist of in the range of about 10 to about 40 weight percent, and/or any range or value therein, of solids (e.g., about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 weight percent).

Turning again to exemplary FIG. 2, after being subjected to the optional pretreatment in the pretreatment system 50, in some embodiments, the pretreated whole stillage can be optionally subjected to enzymatic hydrolysis in an enzymatic hydrolysis system 52. The enzymatic hydrolysis step can be used to break down at least a portion of the starch in the whole stillage into fermentable sugars. In some embodiments, during this treatment, at least 10, 20, or 30 and/or not more than 98, 90, 70, or 60 percent, and/or any range or value therein, of the starch present in the biomass can be broken down into fermentable sugars. In further embodiments, this treatment can break down in the range of 10 to 98 percent of the starch into fermentable sugars. In particular embodiments, this treatment can break down in the range of 10 to 98 percent, and/or any range or value therein, of the starch into fermentable sugars (e.g., about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98 percent).

It should be noted that an enzymatic hydrolysis during a second fermentation as described herein can be more efficient at breaking down the starch into fermentable sugars compared to a hydrolysis step in the primary fermentation. This can be attributed to, at least partly, the lower starch concentrations found in the whole stillage compared to the starch concentrations in the initial biomass feedstock used in the primary fermentation.

In some embodiments, the enzymatic hydrolysis can convert the cellulose portions of the fiber to fermentable sugars and/or convert some of the hemicellulose to sugars. In some embodiments, hexose sugars, such as glucose, may be produced from the cellulose by the enzymatic hydrolysis. In further embodiments, pentose sugars, such as xylose, may be produced from the hemicellulose during the enzymatic hydrolysis.

In some embodiments, during enzymatic hydrolysis, one or more enzymes can be added to the whole stillage to facilitate hydrolyzation of the starch and/or fibers in the whole stillage. In some embodiments, various pH additives can be added such as, for example, ammonia, in order to create an appropriate pH environment for the added enzymes. Different enzymes may be used to hydrolyze the starch, hemicellulose, and cellulose portions of the whole stillage. The enzymes can include but are not limited to a protease, xylanase, cellobiohydrolase, beta-glucosidase cellulase, amylase, hemicellulase, or combinations thereof In some embodiments, the enzymes may be added at a concentration in the range of about 0.001 to 0.5, 0.005 to 0.3, or 0.01 to 0.2 weight percent based on the dry matter of the solids.

As would be readily appreciated in the art, the conditions for enzymatic hydrolysis depend upon the enzymes used and are generally optimized to avoid denaturing the enzymes. For example, in some embodiments, the enzymatic hydrolysis can occur at temperatures in the range of about 100 to about 250° F., about 125 to about 200° F., or about 150 to about 160° F. Additionally, in some embodiments, the enzymatic hydrolysis can occur at pH in the range of about 2 to about 8, about 3 to about 7, or about 4 to about 6. In further embodiments, the enzymatic hydrolysis for the second fermentation can occur at these higher temperatures (e.g., about 100° F. to about 250° F.) compared to the enzymatic hydrolysis step for the primary fermentation step.

In some embodiments, the whole stillage, if subjected to a pretreatment, can be cooled prior to the hydrolysis treatment to a temperature that is more appropriate to facilitate the hydrolysis. In some embodiments, the importance of the enzymatic hydrolysis step in for the breakdown of cellulose and hemicellulose into fermentable sugars can depend on the severity of the pretreatment process. Thus, the less severe the pretreatment process, the more important the enzymatic hydrolysis can be for the breakdown of cellulose and hemicellulose into fermentable sugars

Hemicellulose can be broken down with enzymes that are commercially available. Hemicellulases are generally used to hydrolyze hemicellulose and contain several different enzymes that hydrolyze specific bonds in hemicellulose. Hemicellulases are generally most effective at temperatures in the range of about 155° F. to about 185° F., with reduced activity at fermentation temperatures of about 90° F. to about 95° F. Since hemicellulose composition varies by feedstock, a hemicellulase that is most effective for the particular biomass feedstock or stillage must be selected in embodiments where hydrolysis of the hemicellulose is desired.

In some embodiments, no fermentation of hemicellulose is conducted and as such the enzymatic hydrolysis step may not be required for fermentation. But the quality of the feed products, the ability to dry the feed, the viscosity of the stillage, and yield of oil can be greatly influenced by the hydrolysis of the hemicellulose. Due to its hydrophilic nature, in some embodiments, the hemicellulose can bind liquids, especially water. The bound water, for example, can increase viscosity, thereby increasing pumping requirements, and can increase the energy required to dry the final feed product. In some embodiments, oil can also become bound with the hemicellulose, which decreases oil yields. In addition, hemicellulose may be more digestable by monogastrics when hydrolyzed. Accordingly, depending on the intended use and desired final product(s), the hemicellulose of a stillage or feedstock may or may not be hydrolyzed.

Cellulases are the enzymes that can be used to breakdown cellulose into its derivative sugars. However, cellulose can be difficult to convert to sugars during enzymatic hydrolysis because of its crystalline structure. The glucose is linked to form chains, with crosslinking between the chains. This crosslinking creates much of the difficulty in hydrolyzing cellulose; in effect, it can create a crystalline structure with a relatively small surface area to volume ratio.

Generally, the most effective way of hydrolyzing cellulose is to pretreat it prior to enzymatic hydrolysis as described above in order to rupture the fiber structure, which creates more surface area and decrystallizes the cellulose. Non-pretreated cellulose can have a structure with a very small surface area to volume ratio. This limits the number of areas available for enzymes to attach and liberate glucose from the structure. This determines the effective upper limit for cellulase dosing, thereby limiting the hydrolysis rate. By pretreating the cellulose, the crystalline structure can be disrupted and more areas for attack can be created. The hydrolysis rate can be increased by decreasing polymerization of the cellulose and can be further increased by increased cellulase dosing. Thus, in some embodiments, the cellulose is hydrolyzed.

In some embodiments, the enzymatic hydrolysis of the pretreated cellulose can comprise, consist essentially of or consist of one or more steps, wherein one step comprises cleaving the long chains of glucose from the cellulose using a whole cellulase (e.g., cellulase that is active during the initial steps of breaking the cellulose down”), which randomly hydrolyzes links in the cellulose. Since this action is random, it can create anything from a single glucose unit to a chain that is a few thousand glucose units long. This is generally the cheapest portion of a cellulase enzyme formulation, but since it is random it does not produce free glucose units at a reliable rate. It does, however, create more chains for the next enzymes to act upon. In some embodiments, a further step for hydrolyzing pretreated cellulose can be carried out by cellobiohydrolase. This enzyme can hydrolyze two units of glucose, termed cellobiose, from the end of a cellulose chain. Since this is not a random attack, the rate of production of cellobiose is predictable. In a further embodiment, an additional step for hydrolyzing pretreated cellulose can be carried out by beta-glucosidase. This enzyme can act on the end of a cellulose chain and hydrolyze single units of glucose. The chain can be of any length from two units to thousands of units long. Generally, the best way to cost effectively hydrolyze cellulose is to balance the use of each one of these enzymes. In some embodiments, the cellulase, cellobiohydrolase and beta-glucosidase enzymes can be combined into one or two steps, in any order.

In some embodiments, depending on the nature of the enzyme(s) used, the enzymatic hydrolysis can either be carried out during the subsequent fermentation step described below or as a separate step as described above in a separate tank where the temperature can be held higher so as to facilitate the activity level of the enzymes. Thus, in some embodiments, the enzymatic hydrolysis can be carried out during the fermentation. In other embodiments, the enzymatic step can be carried out separately from the fermentation. The choice of a separate step or a simultaneous enzymatic and fermentation step can depend on the activity of the enzymes used and on viscosity requirements. The whole stillage can become very viscous during the pretreatment steps, especially when cooled to fermentation temperature. In some embodiments, the whole stillage can be cooled to an intermediate temperature where the viscosity is lower after which the enzymatic hydrolysis can be conducted. The whole stillage can then be cooled to fermentation temperatures without excessive viscosity issues.

In some embodiments, the hydrolysis rates can determine the time necessary for the fermentation step. Thus, in some embodiments, the rate of hydrolysis can be increased, thereby reducing the fermentation time. This can be attractive if a fermentation organism is capable of metabolizing the produced sugar as quickly as it is being liberated. The reduced fermentation time reduces the fermentation capacity required, thereby reducing capital costs.

Turning yet again to exemplary FIG. 2, after being subjected to the optional enzymatic hydrolysis in the system 52, the whole stillage and/or wet distiller's grain from the primary fermentation can be subjected to a secondary fermentation in one or more fermentation tanks 54 to produce a secondary fermentation product. In some embodiments, the yeast utilized in the secondary fermentation can include one or more types of yeasts and the choice of yeast(s) can depend on the sugar available for fermentation. For example, Saccharomyces cerevisiae is generally only able to ferment hexose sugars and, therefore, cannot generally use the pentose sugars unlocked from the hemicelluloses. Thus, in some embodiments, two outcomes can generally occur. Either an infectious organism begins to consume the pentose sugars and some of the hexose sugars, or no infection occurs and the pentose sugars remain in solution. In the first case, the final neutral detergent fiber content of the whole stillage produced by the secondary fermentation can be reduced and protein content can be increased, with a slight change in amino acid profile. In the second case, the neutral detergent fiber levels of the whole stillage produced by the secondary fermentation can remain higher, but can exhibit a reduction in the percentage of protein.

Due to the diversity of sugars that can be found in the whole stillage, different combinations of yeasts may need to be utilized in the secondary fermentation to maximize sugar conversion. In some embodiments, the yeasts can include, but are not limited to, Saccharomyces cerevisiae, Pichia stipitis, Candida shehatae, and any combination thereof. In some embodiments, the yeast utilized in the secondary fermentation can be the same or different from the yeast utilized in the primary fermentation step. In one embodiment, the yeast can be Saccharomyces cerevisiae. In other embodiments, the yeast can be S. cerevisiae, P. stipites and C. shehatae.

In some embodiments, the secondary fermentation can occur in the same system and/or vessel as the primary fermentation. In other embodiments, the secondary fermentation can occur in a separate system and/or vessel than the primary fermentation.

The conditions of a secondary fermentation can vary depending on the sugars present in the whole stillage and the effects of the previous pretreatment and hydrolysis steps (if utilized). In some embodiments, a secondary fermentation can occur over a time period in the range of about 12 to about 150, about 24 to about 130, or about 36 to about 110 hours. In some embodiments, about 80 percent of the sugars in the whole stillage can be fermented in at least about 20 hours of fermentation time; however, longer time periods can be used in order to ferment the sugars found in hemicellulose and cellulose. Fermentation usually ceases when the feedstock for the yeasts becomes exhausted. If fermentation is extended beyond this point, then the yeast can go through autolysis and begin to consume their own structural carbohydrates. This can increase the protein levels of the whole stillage byproduct but can have very little influence on final ethanol yields.

In some embodiments, depending on the type of yeasts used, a secondary fermentation can occur at a temperature in the range of about 50 to about 140, about 70 to about 120, or about 80 to about 97 ° F. In some embodiments, a secondary fermentation can occur at a pH in the range of about 3 to about 8, about 3.5 to about 6, or about 4 to about 5. In particular embodiments, the secondary fermentation can occur at a temperature in the range of about 70° F. to about 120° F., and/or any range or value therein (e.g., about 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120° F.) at a pH in the range of about 3.5 to about 6, and/or any range or value therein (e.g., about 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6).

In general, during the first 4 to 6 hours of the fermentation, little to no ethanol can be produced since it is generally during this phase that the yeast are reproducing. In some embodiments, the starch can be the most accessible sugar during these early stages of the process and, therefore, the production of the yeast cells can be generally fueled by the starch. During the post-reproduction phase of fermentation, the yeast can begin to produce ethanol. This can occur as glucose is slowly liberated from the cellulose chains.

Similar to a primary fermentation product, a secondary fermentation product can comprise multiple types of alcohols and other various solid and liquid byproducts. However, ethanol is usually the major component and the most important commercial product produced during the secondary fermentation process. In some embodiments, a secondary fermentation product can comprise, consist essentially of, or consist of at least about 1, 2, 3, or 3.5 and/or not more than about 25, 20, 15, or 10 weight percent, and/or any range or value therein, of ethanol. In further embodiments, a secondary fermentation product can comprise, consist essentially of, or consist of in the range of about 1 to 25, 2 to 20, 3 to 15, or 3.5 to 10 weight percent of ethanol. In particular embodiments, a secondary fermentation product can comprise, consist essentially of, or consist of at least about 1 to about 25 weight percent, and/or any value or range therein, of ethanol (e.g., about 1, 2, 3, 4, 5, 6, 7, 8, 9, 10, 11, 12, 13, 14, 15, 16, 17, 18, 19, 20, 21, 22, 23, 24, 25 weight percent).

Other byproducts included in the secondary fermentation product can include, but are not limited to, glycerol, acetic acid, lactic acid, and carbon dioxide. In some embodiments, the secondary fermentation product can comprise, consist essentially of, or consist of at least about 0.001, 0.005, or 0.01 and/or not more than about 3, 1.5, 0.5, or 0.1 weight percent, and/or any range or value therein, of glycerol. In further embodiments, the secondary fermentation product can comprise, consist essentially of, or consist of in the range of about 0.001 to 1.5, 0.005 to 0.5, 0.01 to 3, or 0.01 to 0.1 weight percent of glycerol. In particular embodiments, the secondary fermentation product can comprise, consist essentially of, or consist of at least about 0.01 to about 3 weight percent, and/or any range or value therein, of glycerol (e.g., about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 1.1, 1.2, 1.3, 1.4, 1.5, 1,6, 1.7, 1.8, 1.9, 2, 2.1, 2.2, 2.3, 2.4, 2.5, 2.6, 2.7, 2.8, 2.9, 3 weight percent). In some embodiments, the secondary fermentation product can comprise, consist essentially of, or consist of at least about 0.0001, 0.001, or 0.01 and/or not more than about 1, 0.5, 0.3, or 0.2 weight percent, and/or any range or value therein, of acetic acid. In further embodiments, the secondary fermentation product can comprise, consist essentially of, or consist of in the range of about 0.0001 to 0.5, 0.001 to 0.3, 0.001 to 1, or 0.01 to 0.2 weight percent of acetic acid. In particular embodiments, the secondary fermentation product can comprise, consist essentially of, or consist of at least about 0.001 to about 1 weight percent, and/or any range or value therein, of acetic acid (e.g., about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1 weight percent). In some embodiments, the secondary fermentation product can comprise, consist essentially of, or consist of at least about 0.001, 0.005, or 0.01 and/or not more than about 2, 1.5, or 1 weight percent, and/or any range or value therein, of lactic acid. In further embodiments, the secondary fermentation product can comprise, consist essentially of, or consist of in the range of about 0.001 to 2, 0.005 to 1.5, 0.01 to 2, or 0.01 to 1 weight percent of lactic acid. In particular embodiments, the secondary fermentation product can comprise, consist essentially of, or consist of at least about 0.01 to about 2 weight percent, and/or any range or value therein, of lactic acid (e.g., about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2 weight percent). It should be noted that the above weight percentages are based on the total weight of the fermentation product unless otherwise noted.

In some embodiments, a secondary fermentation can convert at least a portion of the cellulose and/or hemicellulose in the primary whole stillage into fermentation products. Thus, in some embodiments, a secondary fermentation can convert at least about 30, 40, 50, 60, or 70 percent of the cellulose originally found in the whole stillage into a secondary fermentation product. In further embodiments, a secondary fermentation can convert at least about 30, 40, 50, 60, or 70 percent of the hemicellulose originally found in the whole stillage into a secondary fermentation product. In particular embodiments, a secondary fermentation can convert at least about 30 percent to about 70 percent, and/or any range or value therein (e.g., about 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70 percent) of the cellulose originally found in the primary whole stillage into a secondary fermentation product. In still further embodiments, a secondary fermentation can convert about 30 percent to about 70 percent, and/or any range or value therein (e.g., about 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70 percent) of the hemicellulose originally found in the whole stillage into a secondary fermentation product.

Furthermore, due to the lower starch concentrations of the whole stillage of the present invention as compared with whole stillage produced by art known processes, the ethanol concentration produced during a secondary fermentation can also be low, thereby allowing the yeast to have longer access to the sugars in the whole stillage. Consequently, in some embodiments, this can lead to higher yields of ethanol per bushel of grain. For example, in some embodiments, a secondary fermentation can produce at least about 0.05, 0.15, 0.3, 0.35, or 0.4 and/or not more than about 1.5, 1.0, 0.8, or 0.6 gallons, and/or any range or value therein, of ethanol per bushel of grain. In further embodiments, the secondary fermentation can produce in the range of about 0.15 to 1.5, 0.3 to 1.0, 0.35 to 0.8, 0.4 to 0.6 or 0.05 to 1.5 gallons of ethanol per bushel of grain. In particular embodiments, a secondary fermentation can produce at least about 0.05 to about 1.5 gallons , and/or any range or value therein, of ethanol per bushel of grain (e.g., about 0.05, 0.06, 0.07, 0.08, 0.09, 0.1, 0.2, 0.3, 0.4, 0.5, 0.6, 0.7, 0.8, 0.9, 1, 1.1, 1.2, 1.3, 1.4, 1.5 gallons). In some embodiments, the secondary fermentation can convert at least about 75, 80, 85, 90, or 98 percent of the starch in the whole stillage into the secondary fermentation product.

As shown in exemplary FIG. 2, a secondary fermentation product from the fermentation tanks 54 can be removed and subjected to distillation in one or more distillation columns 56. In some embodiments, in the distillation columns 56, the ethanol can be removed from the secondary fermentation product and treated and purified, for example, as shown in FIG. 1. In some embodiments, the ethanol, and other light alcohols, can exit the top of the columns 56 and be transferred to one or more rectifiers 58 and molecular sieves 60 to remove moisture therefrom. In further embodiments, the final alcohol can then be transferred to one or more ethanol holding tanks 62 where it may be denatured before use as a fuel or fuel additive.

In some embodiments, after removing ethanol and other lighter alcohols from the columns 56, a secondary whole stillage remains in the columns. In further embodiments, the secondary whole stillage can then be transferred to one or more whole stillage holding tanks 64. In some embodiments, the secondary whole stillage can be similar to the whole stillage obtained from the primary fermentation except that it can, for example, have less solids and more protein. Thus, in some embodiments, a secondary whole stillage can comprise, consist essentially of, or consist of at least about 10, 12, 20, or 25 and/or not more than about 60, 50, 40, or 35 weight percent, and/or any range or value therein, of solids. In further embodiments, a secondary whole stillage can comprise, consist essentially of, or consist of in the range of about 10 to 35, 10 to 60, 12 to 50, 20 to 40, or 25 to 35 weight percent of solids. In some embodiments, a secondary whole stillage can comprise, consist essentially of, or consist of at least about 10 to about 35 weight percent, and/or any range or value therein, of solids (e.g., about 10, 11, 12, 13, 14, 15, 16, 17, 18, 19, 20, 21, 22, 23, 24, 25, 26, 27, 28, 29, 30, 31, 32, 33, 34, 35 weight percent of solids). In additional embodiments, a secondary whole stillage can comprise, consist essentially of, or consist of at least about 5, 15, 25, 40 or 65 and/or not more than about 90, 70, 60, or 50 weight percent, and/or any range or value therein, of water. In some embodiments, a secondary whole stillage can comprise, consist essentially of, or consist of in the range of about 5 to 90, 15 to 70, 25 to 60, 40 to 50 or 65 to 90 weight percent of water. In some embodiments, a secondary whole stillage can comprise, consist essentially of, or consist of at least about 65 to about 90 weight percent, and/or any range or value therein, of water (e.g., about 65, 66, 67, 68, 69, 70, 71, 72, 73, 74, 75, 76, 77, 78, 79, 80, 81, 82, 83, 84, 85, 86, 87, 88, 89, 90 weight percent of water).

Furthermore, in some embodiments, due to the conversion of the starch, cellulose, and/or hemicellulose during the second fermentation, a secondary whole stillage can have a reduced amount of these components compared to a whole stillage produced in the primary fermentation. For example, in some embodiments, a secondary whole stillage can comprise, consist essentially of or consist of not more than about 30, 20, 10, or 2 weight percent of starch on a dry matter basis. In some embodiments, a secondary whole stillage can comprise, consist essentially of, or consist of not more than about 30, 15, 10, 5, or 1 weight percent of cellulose and/or hemicellulose on a dry matter basis. In particular embodiments, a secondary whole stillage can comprise, consist essentially of, or consist of not more than about 10 weight percent of starch on a dry matter basis and/or not more than about 30 weight percent of cellulose and/or hemicellulose on a dry matter basis.

As exemplified in FIG. 2, a whole stillage may be passed through one or more centrifuges 66, which can separate the whole stillage into a stream of thin stillage and a stream of wet distiller's grain.

In some embodiments, a thin stillage may be held in one or more tanks 68 and can be returned to the slurry tanks or some other part of the fermentation process that requires water. In some embodiments, a thin stillage can comprise, consist essentially of, or consist of at least about 50, 75, or 85 and/or not more than about 99, 95, or 90 weight percent, and/or any range or value therein, of water. In further embodiments, a thin stillage can comprise, consist essentially of, or consist of in the range of about 50 to 99, 75 to 95, 75 to 99, or 85 to 90 weight percent of water. In particular embodiments, a thin stillage can comprise, consist essentially of, or consist of in the range of about 75 to about 99 weight percent, and/or any range or value therein, of water (e.g., about 75, 76, 77, 78, 79, 80, 81, 82, 83, 84, 85, 86, 87, 88, 89, 90, 91, 92, 93, 94, 95, 96, 97, 98, 99 weight percent). In some embodiments, a thin stillage can comprise, consist essentially of, or consist of at least about 1, 3, or 5 and/or not more than about 25, 20, 15, or 10 weight percent, and/or any range or value therein, of solids. In further embodiments, a thin stillage can comprise, consist essentially of, or consist of in the range of about 1 to 25, 1 to 20, 3 to 15, or 5 to 10 weight percent of solids. In particular embodiments, a thin stillage can comprise, consist essentially of, or consist of in the range of about 1 to about 25 weight percent, and/or any range or value therein, of solids (e.g., about 1, 2, 3, 4, 5, 6, 7, 8, 9, 10, 11, 12, 13, 14, 15, 16, 17, 18, 19, 20, 21, 22, 23, 24, 25 weight percent). In some embodiments, some or all of the thin stillage may also be transferred to one or more evaporators 70 to produce an evaporated thin stillage, which is commonly referred to as “syrup.” In some embodiments, the syrup may be held in one or more tanks 72 and be used as an animal feed additive.

In additional embodiments, the wet distiller's grain, which is often referred to as “wetcake,” may be held in storage facilities 74 and be sold as a livestock feed. In particular embodiments, at least a portion of the wet distiller's grain may be passed through one or more dryers 76 to remove liquid therefrom and thereby produce a dried distiller's grain. In some embodiments, the dried distiller's grain may be stored in one or more tanks 78 and may be used as dry livestock feed. In some embodiments, the syrup from the tanks 66 may also be dehydrated in the dryers 76 in order to form a dried distiller's grain with solubles (“DDGS”).

In some embodiments, a secondary fermentation can produce the dried distiller's grain at a yield of at least about 5, 8, 10, or 11 and/or not more than about 30, 25, 20, or 15 pounds per bushel, and/or any range or value therein, of grain. In some embodiments, a secondary fermentation can produce the dried distiller's grain at a yield in the range of about 5 to 25, 5 to 30, 8 to 25, 10 to 20, or 11 to 15 pounds per bushel of grain. In particular embodiments, a secondary fermentation can produce the dried distiller's grain at a yield of at least about 5 to about 25 pounds per bushel, and/or any range or value therein, of grain (e.g., about 5, 6, 7, 8, 9, 10, 11, 12, 13, 14, 15, 16, 17, 18, 19, 20, 21, 22, 23, 24, 25 pounds per bushel).

In some embodiments, a dried distiller's grain can function as an ideal livestock feed based on its composition. For example, in some embodiments, a dried distiller's grain can comprise, consist essentially of, or consist of at least about 20, 25, or 40 and/or not more than about 70, 60, or 50 weight percent, and/or any range or value therein, of crude protein. In other embodiments, a dried distiller's grain can comprise, consist essentially of, or consist of in the range of about 20 to 70, 20 to 60, 25 to 60, or 40 to 50 weight percent of protein. In particular embodiments, a dried distiller's grain can comprise, consist essentially of, or consist of 20 to 60 weight percent, and/or any value or range therein, of protein (e.g., about 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60 weight percent of protein). In some embodiments, a dried distiller's grain can comprise, consist essentially of, or consist of at least about 1, 2, or 4 and/or not more than about 15, 10, 9, or 8 weight percent, and/or any range or value therein, of crude fat. In further embodiments, the dried distiller's grain can comprise, consist essentially of, or consist of in the range of about 1 to 10, 1 to 15, 2 to 9, or 4 to 8 weight percent of crude fat. In particular embodiments, a dried distiller's grain can comprise, consist essentially of, or consist of about 1 to 15 weight percent, and/or any range or value therein, of crude fat (e.g., about 1, 2, 3, 4, 5, 6, 7, 8, 9, 10, 11, 12, 13, 14, 15 weight percent). In some embodiments, a dried distiller's grain can comprise, consist essentially of, or consist of at least about 0.5, 1, or 2 and/or not more than about 12, 6.5, 6, or 5.6 weight percent, and/or any range or value therein, of crude fiber. In further embodiments, the dried distiller's grain can comprise, consist essentially of, or consist of in the range of about 0.05 to 12, 0.5 to 6.5, 1 to 6, or 2 to 5.6 weight percent of crude fiber. In particular embodiments, a dried distiller's grain can comprise, consist essentially of, or consist of about 0.5 to 12 weight percent, and/or any range or value therein, of crude fiber (e.g., about 0.5, 0.6, 0.7, 0.8, 0.9, 1, 2, 3, 4, 5, 6, 7, 8, 9, 10, 11, 12, 13, 14, 15 weight percent).

In some embodiments, a dried distiller's grain can comprise some of the byproducts derived from the secondary fermentation. For example, in some embodiments, a dried distiller's grain can comprise, consist essentially of, or consist of not more than about 10, 5, 2, 1, or 0.5 weight percent of starch. In further embodiments, a dried distiller's grains can comprise, consist essentially of, or consist of not more than about 40, 10, 7, 5, or 1 weight percent of cellulose and/or hemicellulose. In particular embodiments, a dried distiller's grain can comprise, consist essentially of, or consist of not more than about 10 weight percent of starch and/or not more than about 40 weight percent of cellulose and/or hemicelluloses, and/or any value or range therein. In some embodiments, a dried distiller's grain can comprise, consist essentially of, or consist of at least about 1, 3, or 5 and/or not more than about 35, 22, 18, or 15 weight percent, and/or any range or value therein, of neutral detergent fibers. In further embodiments, the dried distiller's grain can comprise, consist essentially of, or consist of in the range of about 1 to 22, 1 to 35, 3 to 18, or 5 to 15 weight percent of neutral detergent fibers. In particular embodiments, a dried distiller's grain can comprise, consist essentially of, or consist of about 1 to about 35 weight percent, and/or any value or range therein, of neutral detergent fibers (e.g., about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35 weight percent). Neutral detergent fibers can comprise, consist essentially of or consist of cellulose, lignin, and hemicellulose. In further embodiments, a dried distiller's grain can comprise, consist essentially of, or consist of at least about 1, 5, or 9.5 and/or not more than about 40, 30, or 20 weight percent, and/or any range or value therein, of acid detergent fibers. In further embodiments, the dried distiller's grains can comprise, consist essentially of, or consist of in the range of about 1 to 40, 1 to 30, 5 to 30, or 9.5 to 20 weight percent of acid detergent fibers. In particular embodiments, a dried distiller's grain can comprise, consist essentially of, or consist of about 1 to about 30 weight percent, and/or any value or range therein, of acid detergent fibers (e.g., about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 weight percent). Acid detergent fibers can comprise, consist essentially of or consist of cellulose and lignin.

In some embodiments, a primary fermentation and a secondary fermentation step as described herein can be used to convert the majority of the starch, cellulose, and/or hemicellulose originally found in the biomass feedstock into useful products. For example, in some embodiments, a combined output of a primary fermentation and a secondary fermentation can produce at least about 2.65, 2.8, 2.95, or 3.1 and/or not more than about 4, 3.7, 3.5, or 3.3 gallons, and/or any range or value therein, of ethanol per bushel of grain. In further embodiments, a combined output of a primary fermentation and a secondary fermentation can produce in the range of about 2.65 to 3.5, 2.65 to 4, 2.8 to 3.7, 2.95 to 3.5, or 3.1 to 3.3 gallons of ethanol per bushel of grain. In particular embodiments, a combined output of a primary fermentation and a secondary fermentation can produce at least about 2.65 to about 3.5 gallons, and/or any range or value therein, of ethanol per bushel of grain (e.g., about 2.6, 2.65, 2.7, 2.75, 2.8, 2.85, 2.9, 2.95, 3, 3.05, 3.1, 3.15, 3.2, 3.25, 3.3, 3.35, 3.4, 3.45, 3.5 gallons). Furthermore, in some embodiments, combined primary fermentation and secondary fermentation steps can convert at least about 80, 85, 90, or 93 percent of the starch originally found in the biomass feedstock. In some embodiments, combined primary fermentation and secondary fermentation steps can convert at least about 30, 40, 50, 60, 70, 80, 85, 95, or 98 percent of the cellulose and/or hemicellulose originally found in the biomass feedstock. In particular embodiments, combined primary fermentation and secondary fermentation steps can convert at least about 93 percent of the starch originally found in the biomass feedstock, and/or at least about 30 percent of the cellulose and/or hemicellulose originally found in the biomass feedstock

In further embodiments, the process described herein can also improve corn oil recovery by breaking down and fermenting the fiber in the fat-rich germ portion of the kernel. In prior art processes, the oil tends to become trapped within the fiber matrix of the germ, thus making it difficult to remove. Most corn fermentation plants report yields of 15 to 35% of the total oil capable of being recovered. In contrast, by breaking down the fiber as described herein, substantially all of the corn oil can be recovered. In some embodiments, a combined output of a primary fermentation step and a secondary fermentation step as described herein can produce at least about 0.25, 1, 1.25, or 1.5 and/or not more than about 4, 3.0, 2.5, or 2.0 pounds, and/or any range or value therein, of oil per bushel of grain. In some embodiments, a combined output of a primary fermentation and secondary fermentation can produce in the range of about 0.25 to 4, 0.25 to about 2.5, 1 to 3, 1.25 to 2.5, or 1.5 to 2.0 pounds of oil per bushel of grain. In particular, embodiments, a combined output of a primary fermentation and secondary fermentation can produce in the range of about 0.25 to about 2.5 pounds, and/or any value or range therein, of oil per bushel of grain (e.g., about 0.25, 0.3, 0.35, 0.4, 0.45, 0.5, 0.55, 0.6, 0.65, 0.7, 0.75, 0.8, 0.85, 0.9, 0.95, 1, 1.05, 1.1, 1.15, 1.2, 1.25, 1.3, 1.35, 1.4, 1.45, 1.5, 1.55, 1.6, 1.65, 1.7, 1.75, 1.8, 1.85, 1.9, 1.95, 2, 2.05, 2.1, 2.15, 2.2, 2.25, 2.3, 2.35, 2.4, 2.45, 2.5 pounds).

The processes and systems described herein can provide numerous advantages. For example, an advantage of employing a secondary fermentation as described herein is that it can be utilized to maximize ethanol production from the byproducts derived from the primary fermentation step rather than just using the byproducts as animal feed. As a further example of an advantage of the present invention, the byproducts resulting from a secondary fermentation as described herein can be higher in protein and lower in fiber compared to convention fermentation byproducts typically used as animal feed and, therefore, can be easier for monogastrics to digest.

Moreover, although the products of the secondary fermentation can be more expensive to distill due to their higher water contents and lower ethanol contents, these costs can be more than offset by not requiring additional units to separate, evaporate, or liquefy the whole stillage prior to the secondary fermentation. In addition, in some embodiments, since the whole stillage used in the secondary fermentation as described herein has already been subjected to a distillation process during the primary fermentation, at least a portion of the water therein has already been removed by this previous step. Thus, the total volume of water going through the secondary fermentation may be decreased.

Further advantages of the process and system described herein include that the whole stillage and thin stillage produced after the secondary fermentation can have a higher solids content compared to the corresponding byproducts produced in conventional processes. This can be due to the high solids content in the initial biomass feedstock and the two distillation steps discussed herein, which can remove a significant portion of the water from these products.

The embodiments of the invention described above are to be used as illustration only, and should not be used in a limiting sense to interpret the scope of the present invention. Modifications to the exemplary embodiments, set forth above, could be readily made by those skilled in the art without departing from the spirit of the present invention.

The inventors hereby state their intent to rely on the Doctrine of Equivalents to determine and assess the reasonably fair scope of the present invention as it pertains to any apparatus not materially departing from but outside the literal scope of the invention as set forth in the following claims.

It should be understood that the following is not intended to be an exclusive list of defined terms. Other definitions may be provided in the foregoing description, such as, for example, when accompanying the use of a defined term in context.

All publications and patent applications mentioned in this specification are indicative of the level of skill of those skilled in the art that this invention pertains. Further, publications, patent applications, patents, and other references mentioned herein are incorporated by reference in their entireties for the teachings relevant to the sentence and/or paragraph in which the reference is presented.

As used in the description of the embodiments of the invention and the appended claims, the singular forms “a,” “an,” and “the” are intended to include the plural forms as well, unless the context clearly indicates otherwise.

The term “about,” as used herein when referring to a measurable value such as an amount of a compound, dose, time, temperature, and the like, refers to variations of 20%, 10%, 5%, 1%, 0.5%, or even 0.1% of the specified amount.

As used herein, the term “and/or,” when used in a list of two or more items, means that any one of the listed items can be employed by itself or any combination of two or more of the listed items can be employed. For example, if a composition is described as containing components A, B, and/or C, the composition can contain A alone; B alone; C alone; A and B in combination; A and C in combination, B and C in combination; or A, B, and C in combination.

As used herein, the terms “comprising,” “comprises,” and “comprise” are open-ended transition terms used to transition from a subject recited before the term to one or more elements recited after the term, where the element or elements listed after the transition term are not necessarily the only elements that make up the subject.

As used herein, the transitional phrase “consisting essentially of (and grammatical variants) means that the scope of a claim is to be interpreted to encompass the specified materials or steps recited in the claim and those that do not materially alter the basic and novel characteristic(s)” of the claimed invention. Thus, the term “consisting essentially of” when used in a claim of this invention is not intended to be interpreted to be equivalent to “comprising.”

As used herein, the terms “having,” “has,” and “have” have the same open-ended meaning as “comprising,” “comprises,” and “comprise” provided above.

As used herein, the terms “including,” “include,” and “included” have the same open-ended meaning as “comprising,” “comprises,” and “comprise” provided above.

As used herein, references to “one embodiment,” “an embodiment,” or “embodiments” mean that the feature or features being referred to are included in at least one embodiment of the technology. Separate references to “one embodiment,” “an embodiment,” or “embodiments” in this description do not necessarily refer to the same embodiment but are also not mutually exclusive of one another unless so stated and/or except as will be readily apparent to those skilled in the art from the description. Thus, the present invention can include a variety of combinations and/or integrations of the embodiments described herein.

The present description uses numerical ranges to quantify certain parameters relating to the invention. It should be understood that when numerical ranges are provided, such ranges are to be construed as providing literal support for claim limitations that only recite the lower value of the range as well as claim limitations that only recite the upper value of the range. For example, a disclosed numerical range of 10 to 100 provides literal support for a claim reciting “greater than 10” (with no upper bounds) and a claim reciting “less than 100” (with no lower bounds).

The invention will now be described with reference to the following examples. It should be appreciated that these examples are not intended to limit the scope of the claims to the invention, but are rather intended to be exemplary of certain embodiments. Any variations in the exemplified methods that occur to the skilled artisan are intended to fall within the scope of the invention.

Accordingly, a method for producing a biomass-derived product is provided, the method comprising: subjecting a primary whole stillage to fermentation to thereby produce a fermentation product comprising a secondary whole stillage and ethanol, wherein the whole stillage has a starch content of at least 15 weight percent on a dry matter basis. In some embodiments, the primary whole stillage comprises about 20 to about 80 weight percent of starch on a dry matter basis. In some embodiments, the primary whole stillage comprises about 10 to about 60 weight percent of solids. In some embodiments, the primary whole stillage comprises about 5 to about 90 weight percent of water. In some embodiments, the primary whole stillage comprises about 5 to about 30 weight percent of cellulose on a dry basis. In some embodiments, the primary whole stillage of the primary fermentation comprises about 1 to about 10 weight percent of short chain sugars having a degree of polymerization of about 4 to about 20. In some embodiments, the fermentation occurs in the presence of a yeast, wherein the yeast can be, but is not limited to, Saccharomyces cerevisiae, Pichia stipitis, and/or Candida shehatae. In some embodiments, the ethanol of the fermentation product is in a concentration of about 1 to about 25 weight percent. In some embodiments, the fermentation produces about 0.15 to about 0.6 gallons of ethanol per bushel of grain. In some embodiments, the fermentation converts about 30 to about 70 percent of the cellulose in the primary whole stillage into a secondary fermentation product. In some embodiments, the fermentation converts about 75 to about 90 percent of the starch in the primary whole stillage.

In some embodiments, the secondary whole stillage comprises about 2 to about 30 weight percent of starch on a dry matter basis. In some embodiments, the secondary whole stillage comprises about 10 to about 60 weight percent of solids. In some embodiments, the secondary whole stillage comprises about 5 to about 90 weight percent of water. In some embodiments, the secondary whole stillage comprises about 1 to about 15 weight percent of cellulose on a dry basis.

In some embodiments, the secondary whole stillage can be separated into a distiller's grain and a thin stillage. In some embodiments, the distiller's grain can be dried to form a dried distiller's grain. In some embodiments, the distiller's grain comprises about 20 to about 70 weight percent of crude protein. In some embodiments, the distiller's grain comprises about 1 to about 10 weight percent of crude fat. In some embodiments, the distiller's grain comprises about 0.5 to about 6.5 weight percent of crude fiber. In some embodiments, the distiller's grain comprises about 1 to about 22 weight percent of neutral detergent fibers. In some embodiments, the distiller's grain comprises about 1 to about 40 weight percent of acid detergent fibers. In some embodiments, the distiller's grain comprises about 0.5 to about 15 weight percent of cellulose on a dry basis. In some embodiments, the distiller's grain comprises about 0.5 to about 5 weight percent of starch. In some embodiments, a secondary fermentation produces the distiller's grain at a yield of about 5 to about 30 pounds per bushel of grain.

In some embodiments, the fermentation product of the primary whole comprises about 0.001 to about 1.5 weight percent of glycerol. In some embodiments, the fermentation product comprises about 0.0001 to about 0.5 weight percent of acetic acid. In some embodiments, the fermentation product comprises about 0.001 to about 2 weight percent of lactic acid.

In some embodiments, the primary whole stillage can be pretreated prior to the fermentation to produce a pretreated whole stillage. In some embodiments, the pretreatment comprises acid hydrolysis, enzymatic hydrolysis, steam explosion, alkaline treatment, drying, grinding, or a combination thereof. In some embodiments, the grinding comprises wet milling or dry milling. In some embodiments, the pretreating comprises hydrolyzing the whole stillage to form a hydrolyzed whole stillage. In some embodiments, the whole stillage subjected to the fermentation comprises the hydrolyzed whole stillage. In some embodiments, the hydrolyzing occurs in the presence of at least one enzyme, wherein the enzyme comprises a protease, xylanase, cellobiohydrolase, beta-glucosidase cellulase, amylase, hemicellulase, or any combination thereof In some embodiments, the pretreated whole stillage comprises about 1 to about 50 weight percent of starch on a dry matter basis. In some embodiments, the pretreated whole stillage comprises about 5 to about 50 weight percent of solids. In some embodiments, the pretreated whole stillage comprises at least about 2 to about 25 weight percent of cellulose on a dry matter basis.

In further embodiments, a method for producing a biomass-derived product is provided, the method comprising: (a) subjecting a biomass feedstock to a primary fermentation to thereby produce a primary fermentation product comprising a primary whole stillage and ethanol, wherein the biomass feedstock has a starch content of at least 22 weight percent; and (b) subjecting the primary whole stillage to a secondary fermentation to thereby produce a secondary fermentation product comprising a secondary whole stillage and ethanol. In some embodiments, the biomass feedstock and primary whole stillage have a starch ratio of about 2:1 to about 100:1. In some embodiments, the primary fermentation product comprises about 0.1 to about 5 weight percent of glycerol. In some embodiments, the primary fermentation product comprises about 0.001 to about 0.5 weight percent of acetic acid. In some embodiments, the primary fermentation product comprises about 0.001 to about 2 weight percent of lactic acid. In some embodiments, the primary fermentation product comprises about 7 to about 40 weight percent of ethanol. In some embodiments, the primary fermentation of step (a) produces about 1.3 to about 3.5 gallons of ethanol per bushel of grain. In some embodiments, the primary whole stillage comprises about 5 to about 80 weight percent of starch on a dry matter basis. In some embodiments, the primary whole stillage comprises about 10 to about 60 weight percent of solids. In some embodiments, the primary whole stillage comprises about 5 to about 90 weight percent of water. In some embodiments, the primary whole stillage comprises about 5 to about 30 weight percent of cellulose on a dry basis. In some embodiments, the primary whole stillage of the primary fermentation comprises about 1 to about 10 weight percent of short chain sugars having a degree of polymerization of about 4 to about 20. In some embodiments, the primary fermentation of step (a) occurs in the presence of a yeast, wherein the yeast can be, but is not limited to, Saccharomyces cerevisiae, Pichia stipitis, and/or Candida shehatae. In some embodiments, the primary fermentation (a) and the secondary fermentation (b) can occur in the same and/or separate vessels or systems.

In some embodiments, the primary fermentation converts about 50 to about 95 percent of the starch originally found in the biomass feedstock.

In some embodiments, prior to the secondary fermentation, primary fermentation product can be distilled to separate the primary whole stillage and ethanol. In some embodiments, the primary whole stillage comprises about 5 to about 90 weight percent of water. In some embodiments, the primary whole stillage comprises about 10 to about 60 weight percent of solids. In some embodiments, the whole stillage of the primary fermentation product comprises about 1 to about 10 weight percent of sugars having a degree of polymerization of about 4 to about 20.

In some embodiments, the biomass feedstock subjected to a primary fermentation comprises about 30 to about 90 weight percent of starch. In some embodiments, the biomass feedstock comprises a solids content of about 35 to about 90 weight percent. In some embodiments, the biomass feedstock comprises a grain. In some embodiments, the biomass feedstock comprises about 20 to about 90 weight percent grain. In some embodiments, the grain comprises a ground grain. In some embodiments, the grain comprises barley, rye, wheat, oats, sorghum, milo, canola, corn, buckwheat, or any combination thereof. In some embodiments, the biomass feedstock comprises water. In some embodiments, the biomass feedstock comprises about 10 to about 90 weight percent of water. In some embodiments, about 5 to about 95 percent of the water can be from a thin stillage. In some embodiments, the biomass feedstock comprises a thin stillage.

In some embodiments, biomass feedstock subjected to a primary fermentation comprises about 0.5 to about 20 weight percent of the thin stillage. In some embodiments, the thin stillage comprises about 50 to about 99 weight percent of water. In some embodiments, the thin stillage comprises a solids content of about 1 to about 20 weight percent. In some embodiments, the solids in the thin stillage comprise about 0.5 to about 20 weight percent of the biomass feedstock. In some embodiments, the biomass feedstock comprises whole stillage recovered from other fermentation processes.

In some embodiments, prior to the primary fermentation, the biomass feedstock can be pretreated to yield a pretreated biomass feedstock. In some embodiments, the pretreated biomass feedstock is the biomass feedstock in the primary fermentation of step (a). In some embodiments, the pretreatment comprises enzymatic hydrolysis. In some embodiments, the secondary fermentation product comprises about 1 to about 25 weight percent of ethanol.

In some embodiments, the secondary fermentation produces about 0.15 to about 1.5 gallons of ethanol per bushel of grain. In some embodiments, the secondary fermentation converts about 30 to about 70 percent of the cellulose in the primary whole stillage into the secondary fermentation product. In some embodiments, the secondary fermentation converts about 75 to about 90 percent of the starch in the primary whole stillage. In some embodiments, the secondary fermentation occurs in the presence of a yeast, wherein the yeast can be, but is not limited, to Saccharomyces cerevisiae, Pichia stipitis, and/or Candida shehatae.

In some embodiments, the secondary whole stillage comprises about 2 to about 30 weight percent of starch on a dry matter basis. In some embodiments, the secondary whole stillage comprises about 10 to about 60 weight percent of solids. In some embodiments, the secondary whole stillage comprises about 5 to about 90 weight percent of water. In some embodiments, the secondary whole stillage comprises about 1 to about 15 weight percent of cellulose on a dry basis.

In some embodiments, the secondary whole stillage can be separated into a distiller's grain and a thin stillage. In some embodiments, the distiller's grain can be dried to form a dried distiller's grain. In some embodiments, the distiller's grain comprises about 20 to about 70 weight percent of crude protein. In some embodiments, the distiller's grain comprises about 1 to about 10 weight percent of crude fat. In some embodiments, the distiller's grain comprises about 0.5 to about 6.5 weight percent of crude fiber. In some embodiments, the distiller's grain comprises about 1 to about 22 weight percent of neutral detergent fibers. In some embodiments, the distiller's grain comprises about 1 to about 40 weight percent of acid detergent fibers. In some embodiments, the distiller's grain comprises about 1 to about 10 weight percent of cellulose on a dry matter basis. In some embodiments, herein the distiller's grain comprises about 0.5 to about 5 weight percent of starch.

In some embodiments, the secondary fermentation produces the distiller's grain at a yield of at least 5 to about 30 pounds per bushel of grain. In some embodiments, the secondary fermentation product comprises about 0.001 to about 1.5 weight percent of glycerol. In some embodiments, the secondary fermentation product comprises about 0.0001 to about 0.5 weight percent of acetic acid. In some embodiments, the secondary fermentation product comprises about 0.001 to about 2 weight percent of lactic acid.

In some embodiments, the primary whole stillage can be pretreated prior to the secondary fermentation of step (b). In some embodiments, the pretreating comprises subjecting the primary whole stillage to an acid hydrolysis, enzymatic hydrolysis, drying, alkaline treatment, steam explosion, grinding, or any combination thereof. In some embodiments, the grinding comprises wet milling or dry milling. In some embodiments, the pretreating comprises hydrolyzing the primary whole stillage to form a hydrolyzed whole stillage.

In some embodiments, the hydrolyzed whole stillage is the primary whole stillage in the secondary fermentation of step (b). In some embodiments, the hydrolyzing occurs in the presence of an enzyme and the enzyme comprises a protease, xylanase, cellobiohydrolase, beta-glucosidase cellulase, amylase, hemicellulase, or any combination thereof. In some embodiments, the hydrolyzed whole stillage comprises about 1 to about 50 weight percent of starch on a dry matter basis. In some embodiments, the hydrolyzed whole stillage comprises about 5 to about 50 weight percent of solids. In some embodiments, the hydrolyzed whole stillage comprises about 2 to about 25 weight percent of cellulose on a dry basis.

In some embodiments, the combined output of the primary fermentation of step (a) and the secondary fermentation of step (b) produce about 2.65 to about 4 gallons of ethanol per bushel of grain. In some embodiments, the combined output of the primary fermentation of step (a) and the secondary fermentation of step (b) produce about 0.25 to about 4 pounds of oil per bushel of grain. In some embodiments, the primary fermentation and secondary fermentation convert about 80 to about 93 percent of the starch originally found in the biomass feedstock.

In further embodiments, a method for producing a biomass-derived product is provided, the method comprising: (a) subjecting a biomass feedstock to a primary fermentation to thereby produce a primary fermentation product comprising a primary whole stillage and ethanol, wherein the biomass feedstock has a starch content of at least 20 weight percent and the primary whole stillage has a starch content of at least 15 weight percent on a dry matter basis; (b) pretreating the primary whole stillage to thereby produce a pretreated whole stillage; and (c) subjecting the pretreated whole stillage to a secondary fermentation to thereby produce a secondary fermentation product comprising a secondary whole stillage and ethanol. the biomass feedstock and primary whole stillage have a starch ratio of about 2:1 to about 100:1. In some embodiments, the primary fermentation product comprises about 0.1 to about 5 weight percent of glycerol. In some embodiments, the primary fermentation product comprises about 0.001 to about 0.5 weight percent of acetic acid. In some embodiments, the primary fermentation product comprises about 0.001 to about 2 weight percent of lactic acid. In some embodiments, the primary fermentation product comprises about 7 to about 40 weight percent of ethanol. In some embodiments, the primary fermentation (step (a)) produces about 1.3 to about 3.5 gallons of ethanol per bushel of grain. In some embodiments, the primary fermentation (a), pretreating (b) and the secondary fermentation (c) can occur in the same and/or separate vessels or systems

In some embodiments, the primary whole stillage comprises about 5 to about 80 weight percent of starch on a dry matter basis. In some embodiments, the primary whole stillage comprises about 10 to about 60 weight percent of solids. In some embodiments, the primary whole stillage comprises about 5 to about 90 weight percent of water. In some embodiments, the primary whole stillage comprises about 5 to about 30 weight percent of cellulose on a dry basis. In some embodiments, the primary whole stillage comprises about 1 to about 10 weight percent of short chain sugars having a degree of polymerization of about 4 to about 20. In some embodiments, the primary fermentation converts about 75 to about 95 percent of the starch originally found in the biomass feedstock. In some embodiments, the primary fermentation of step (a) occurs in the presence of a yeast, wherein the yeast can be, but is not limited, to Saccharomyces cerevisiae. In some embodiments, prior to the pretreatment (step (b)), the primary fermentation product is distilled to separate the primary whole stillage and ethanol.

In some embodiments, the primary whole stillage comprises about 5 to about 50 weight percent of water. In some embodiments, the primary whole stillage comprises about 10 to about 60 weight percent of solids. In some embodiments, the primary whole stillage of the primary fermentation product comprises about 1 to about 10 weight percent of sugars having a degree of polymerization of about 4 to about 20.

In some embodiments, the biomass feedstock subjected to a primary fermentation comprises about 30 to about 90 weight percent of starch. In some embodiments, the biomass feedstock comprises a solids content of about 35 to about 90 weight percent. In some embodiments, the biomass feedstock comprises a grain. In some embodiments, the biomass feedstock comprises about 20 to about 90 weight percent grain. In some embodiments, the grain comprises a ground grain. In some embodiments, the grain comprises barley, rye, wheat, oats, sorghum, milo, canola, corn, buckwheat, or any combination thereof. In some embodiments, the biomass feedstock subjected to the primary fermentation comprises water. In some embodiments, the biomass feedstock comprises about 10 to about 90 weight percent of water. In some embodiments, about 5 to about 95 percent of the water is from a thin stillage. In some embodiments, the biomass feedstock comprises a thin stillage. In some embodiments, the biomass feedstock comprises about 0.5 to about 20 weight percent of the thin stillage. In some embodiments, the thin stillage comprises about 50 to about 99 weight percent of water. In some embodiments, the thin stillage comprises a solids content of about 1 to about 20 weight percent. In some embodiments, the solids in the thin stillage comprise about 0.5 to about 20 weight percent of the biomass feedstock. In some embodiments, the biomass feedstock comprises whole stillage recovered from other fermentation processes. In some embodiments, prior to the primary fermentation, the biomass feedstock can be pretreated to yield a pretreated biomass feedstock.

In some embodiments, the pretreated biomass feedstock is the biomass feedstock in the primary fermentation of step (a). In some embodiments, the pretreatment comprises enzymatic hydrolysis.

In some embodiments, the secondary fermentation product comprises about 1 to about 25 weight percent of ethanol. In some embodiments, the secondary fermentation produces about 0.15 to about 1.5 gallons of ethanol per bushel of grain. In some embodiments, the secondary fermentation converts about 30 to about 70 percent of the cellulose in the pretreated whole stillage into the secondary fermentation product. In some embodiments, the secondary fermentation converts about 75 to about 90 percent of the starch in the pretreated whole stillage. In some embodiments, the secondary fermentation occurs in the presence of a yeast, wherein the yeast can be, for example, Saccharomyces cerevisiae.

In some embodiments, the secondary whole stillage comprises about 2 to about 30 weight percent of starch on a dry matter basis. In some embodiments, the secondary whole stillage comprises about 10 to about 60 weight percent of solids. In some embodiments, the secondary whole stillage comprises about 5 to about 90 weight percent of water. In some embodiments, the secondary whole stillage comprises about 1 to about 15 weight percent of cellulose on a dry basis. In some embodiments, the secondary whole stillage can be separated into a distiller's grain and a thin stillage. In some embodiments, the distiller's grain can be dried to form a dried distiller's grain. In some embodiments, the distiller's grain comprises about 20, to about 70 weight percent of crude protein. In some embodiments, the distiller's grain comprises about 1 to about 10 weight percent of crude fat. In some embodiments, the distiller's grain comprises about 0.5 to about 6.5 weight percent of crude fiber. In some embodiments, the distiller's grain comprises about 1 to about 22 weight percent of neutral detergent fibers. In some embodiments, the distiller's grain comprises about 1 to about 40 weight percent of acid detergent fibers. In some embodiments, the distiller's grain comprises about 1 to about 10 weight percent of cellulose on a dry matter basis. In some embodiments, the distiller's grain comprises about 0.5 to about 5 weight percent of starch.

In some embodiments, the secondary fermentation produces the distiller's grain at a yield of about 5 to about 30 pounds per bushel of grain. In some embodiments, the secondary fermentation product comprises about 0.001 to about 1.5 weight percent of glycerol. In some embodiments, the secondary fermentation product comprises about 0.0001 to about 0.5 weight percent of acetic acid. In some embodiments, the secondary fermentation product comprises about 0.001 to about 2 weight percent of lactic acid.

In some embodiments, pretreating the primary whole stillage (step (b)) comprises subjecting the primary whole stillage to acid hydrolysis, enzymatic hydrolysis, drying, steam explosion, grinding, or any combination thereof. In some embodiments, the grinding comprises wet milling or dry milling.

In some embodiments, pretreating the primary whole stillage (step (b)) comprises hydrolyzing the primary whole stillage to form a hydrolyzed whole stillage. In some embodiments, the hydrolyzed whole stillage is the pretreated whole stillage in the secondary fermentation of step (c). In some embodiments, the hydrolyzing occurs in the presence of an enzyme, wherein the enzyme comprises a protease, xylanase, cellobiohydrolase, beta-glucosidase cellulase, amylase, hemicellulase, or combinations thereof.

In some embodiments, pretreated whole stillage comprises about 1 to about 50 weight percent of starch on a dry matter basis. In some embodiments, pretreated whole stillage comprises about 5 to about 50 weight percent of solids. In some embodiments, pretreated whole stillage comprises about 2 to about 25 weight percent of cellulose on a dry basis.

In some embodiments, the combined output of the primary fermentation (a) and the secondary fermentation (b) produces about 2.65 to about 4 gallons of ethanol per bushel of grain. In some embodiments, the combined output of the primary fermentation (a) and the secondary fermentation (b) produces about 0.25 to about 4 pounds of oil per bushel of grain. In some embodiments, the primary fermentation and secondary fermentation convert about 80 to about 93 percent of the starch originally found in the biomass feedstock.

1. A method for producing a biomass-derived product, the method comprising:
subjecting a primary whole stillage to fermentation to thereby produce a fermentation product comprising a secondary whole stillage and ethanol, wherein the whole stillage has a starch content of at least 15 weight percent on a dry matter basis.

2. The method of claim 1, wherein the primary whole stillage comprises about 20 to about 80 weight percent of starch on a dry matter basis, about 10 to about 60 weight percent of solids, and/or about 5 to about 90 weight percent of water.

3. The method of claim 1, wherein the primary whole stillage comprises about 5 to about 30 weight percent of cellulose on a dry basis.

4. The method of claim 1, wherein the primary whole stillage of the primary fermentation comprises about 1 to about 10 weight percent of short chain sugars having a degree of polymerization of about 4 to about 20.

5. The method of claim 1, wherein the fermentation occurs in the presence of a yeast, optionally, wherein the yeast is Saccharomyces cerevisiae, Pichia stipitis, and/or Candida shehatae.

6. The method of claim 1, wherein the fermentation produces about 0.15 to about 0.6 gallons of ethanol per bushel of grain and/or the ethanol of the fermentation product is in a concentration of about 1 to about 25 weight percent.

7. The method of claim 3, wherein the fermentation converts about 30 to about 70 percent of the cellulose in the primary whole stillage into a secondary fermentation product and/or converts about 75 to about 90% of the starch in the primary whole stillage.

8. (canceled)

9. The method of claim 1, wherein the fermentation product comprises about 0.001 to about 1.5 weight percent of glycerol, about 0.0001 to about 0.5 weight percent of acetic acid, and/or about 0.001 to about 2 weight percent of lactic acid.

10. The method of claim 1, wherein the secondary whole stillage comprises about 2 to about 30 weight percent of starch on a dry matter basis and/or about 10 to about 60 weight percent of solids, optionally wherein the secondary whole stillage further comprises about 5 to about 90 weight percent of water and/or about 1 to about 15 weight percent of cellulose on a dry basis.

11. (canceled)

12. The method of claim 1, further comprising separating secondary whole stillage into a distiller's grain and a thin stillage.

13. The method of claim 12, further comprising drying the distiller's grain to form a dried distiller's grain.

14. The method of claim 13, wherein the dried distiller's grain comprises about 20 to about 70 weight percent of crude protein, about 1 to about 10 weight percent of crude fat, and/or about 0.5 to about 6.5 weight percent of crude fiber, optionally wherein the dried distiller's grain further comprises about 1 to about 22 weight percent of neutral detergent fibers, and/or about 1 to about 40 weight percent of acid detergent fibers.

15. (canceled)

16. The method of claim 13, wherein the dried distiller's grain comprises about 0.5 to about 15 weight percent of cellulose on a dry basis and/or about 0.5 to about 5 weight percent of starch.

17. (canceled)

18. The method of claim 1, further comprising pretreating the primary whole stillage prior to the fermentation to produce a pretreated whole stillage.

19. The method of claim 18, wherein the pretreatment comprises acid hydrolysis, enzymatic hydrolysis, steam explosion, alkaline treatment, drying, grinding, or a combination thereof.

20. The method of claim 19, wherein the grinding comprises wet milling or dry milling.

21. The method of claim 18, wherein the pretreating comprises hydrolyzing the primary whole stillage to form a hydrolyzed whole stillage.

22. The method of claim 21, wherein the whole stillage subjected to the fermentation comprises the hydrolyzed whole stillage.

23. The method of claim 22, wherein the hydrolyzing occurs in the presence of at least one enzyme, wherein the enzyme comprises a protease, xylanase, cellobiohydrolase, beta-glucosidase cellulase, amylase, hemicellulase, or any combination thereof.

24. The method of claim 18, wherein the pretreated whole stillage comprises about 1 to about 50 weight percent of starch on a dry matter basis, about 5 to about 50 weight percent of solids, and/or about 2 to about 25 weight percent of cellulose on a dry matter basis.
